(19) United States
(12) Patent Application Publication
(10) Pub. No.: US 2026/0224105 A1
(43) Pub. Date: Aug. 6, 2026

(54) DETECTING ENVIRONMENTAL ATTRIBUTES FOR EYE HEALTH

(71) Applicant: Samsung Electronics Co., Ltd., Suwon-si (KR)

(72) Inventors: Maxim Latrouitte Armstrong, Pacifica, CA (US); Subramaniam Venkatraman, Lafayette, CA (US)

(21) Appl. No.: 19/321,833

(22) Filed: Sep. 8, 2025

Related U.S. Application Data
(60) Provisional application No. 63/754,430, filed on Feb. 5, 2025.

(51) Int. Cl. A61B 3/113; A61B 3/14; G06T 5/20; G06T 7/00; G06T 7/246; H04N 23/67

(52) U.S. Cl. CPC A61B 3/113; A61B 3/14; G06T 5/20; G06T 7/0012; G06T 7/246; H04N 23/67; G06T 2207/30041

(57) ABSTRACT

In one embodiment, a method includes estimating a gaze of a wearer of a device that includes an outward facing camera. The method further includes focusing the camera based on the estimated gaze of the wearer; capturing, by the focused camera, one or more focused images of an environment of the wearer; and determining, from at least one of the one or more focused images of the environment of the wearer, one or more visual features of the environment of the wearer related to the eye health of the wearer.

### PRIORITY CLAIM

This application claims the benefit under 35 U.S.C. § 119 of U.S. Provisional Patent Application No. 63/754,430 filed Feb. 5, 2025, which is incorporated by reference herein.

### TECHNICAL FIELD

This application generally relates to detecting environmental attributes for eye health.

### BACKGROUND

There exist a number of important eye health metrics that tend to change over long time scales, such that short-term changes are imperceptible and difficult to diagnose. For example, myopia (nearsightedness) is associated with significant morbidity, including increases in vision-threatening diseases such as retinal detachment, glaucoma, cataracts, and macular degeneration. Myopia is becoming more prevalent, as least partly due to the increase in near-vision activities such as using electronic screens or working on objects at close distances (e.g., within arm's length). In addition, an increased prevalence in time spent indoors results in increased exposure to low-light conditions and relatively more uniform visual environments.

Myopia progression is relatively slow, as associated elongation of the eyeball occurs relatively slowly, typically over a period of several years. The physical changes associated with myopia typically occur in the early portion of a human's lifespan, e.g., prior to around age 30.

### BRIEF DESCRIPTION OF THE DRAWINGS

FIG. 1 illustrates an example method for detecting one or more visual features of the environment of the wearer related to the eye health of the wearer.

FIG. 2 illustrates an example technique that implements the example method of FIG. 1.

FIG. 3 illustrates an example of a Fourier transform of a particular image and filtering based on a band of spatial frequencies.

FIG. 4 illustrates example UIs that provides example eye-health related information determined from the wearer's visual environment.

FIG. 5 illustrates an example computing system.

### DESCRIPTION OF EXAMPLE EMBODIMENTS

There exist a number of important eye health metrics that tend to change over long time scales, such that short-term changes are imperceptible and difficult to diagnose. For example, the progression of myopia is both slow and imperceptible over relatively short time periods, and therefore it is difficult for a person to determine whether myopia is progressing, e.g., in the intervening periods between clinic visits. In addition, as explained below, many of the visual features that are related to myopia progression and the occurrence of those features (e.g., the relative contrast between features in central vs. peripheral vision) are difficult if not impossible for a person to discern in their own environment. As a result, the progression of myopia and occurrence of the underlying environmental conditions that cause myopia are not identifiable as person goes about their daily life.

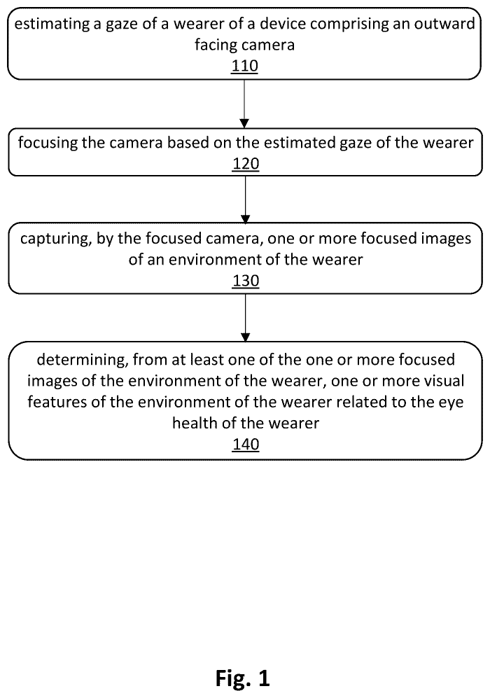

FIG. 1. illustrates an example method for detecting one or more visual features of the environment of the wearer related to the eye health of the wearer. Such eye-health conditions include myopia, dry eyes, etc. This disclosure uses myopia as an example of an eye-health condition, given its impact and global importance. For instance, an individual's personal, daily visual environment can be evaluated for conditions that tend to cause myopia, and this information can be relayed to the individual, to a health professional (e.g., the individual's doctor), and/or used to provide recommendations regarding the person's visual environment, both in real time and over time.

Step 110 of the example method of FIG. 1 includes estimating a gaze of a wearer of a device that includes an outward facing camera. In particular embodiments, the device worn by a wearer may be a head-worn device, such as a pair of glasses or a head-mounted device (HMD). Other devices may be worn on other portions of the body and include an outward-facing camera. The outward-facing camera faces away from the wearer's body, in the general direction of the wearer's view, and one advantage of embodiments that use a head-worn device is that the outward-facing camera tracks the wearer's view as the wearer turns their head.

In particular embodiments, estimating a gaze of the wearer includes capturing, by the outward facing camera of the device worn by the wearer, an initial image of an environment of the wearer, and then estimating a portion of the initial image that corresponds to the gaze of the wearer. As explained herein, an initial image may be captured periodically (e.g., after a predetermined amount of time) and/or may be triggered based on changes in the wearer's visual environment. Here, capturing the initial image does not necessarily require storing that initial image, although some embodiments may do so. Instead, capturing the initial image (which can include capturing multiple initial images, e.g., in an iterative autofocus process) just requires the camera to detect an image of the environment via its sensing capabilities. The initial image may be stored only transiently to focus the camera, e.g., during an autofocus process, as described below.

In embodiments that estimate a gaze of the wearer by capturing an initial image, then the initial image is captured at a particular focus setting for the camera. This initial focus setting may not correspond to the gaze of the wearer, e.g., the focus may be on an object at a particular depth, while the wearer may in fact by looking at a different object, which may be at a different depth. Such embodiments therefore estimate which portion of the initial image corresponds to the wearer's gaze, so that subsequent images captured by the camera can accurately capture features of the visual environment of the wearer.

In particular embodiments, estimating a portion of the initial image that corresponds to a gaze of the wearer may include determining a relatively central portion of the image (e.g., the central ⅓ of the image, or a central angular portion such as the central 10-18 degrees about the center of the image, etc.) and focusing the camera on features that correspond to that region. In such embodiments, the system assumes that the wearer is looking relatively straight ahead, which is typically a good approximation—particularly for head-worn devices, as people typically don't spend a large amount of time holding their gaze to the side.

For example, particular embodiments may determine an average difference between the wearer's gaze and the optical axis of the camera. This technique may be performed from population measurements (e.g., by averaging the difference across many wearers) or by a wearer-specific calibration (e.g., having a wearer look at various objects in a scene while capturing images of the scene, and measuring the difference between the camera axis and the object's location in the image). Then, the angle of the wearer's gaze for subsequent images may be estimated using this axis-related information.

In particular embodiments, estimating a portion of the initial image that corresponds to a gaze of the wearer may include detecting an object in the initial image. The presence of certain objects in the environment of a person tend to be the object of the wearer's gaze. For example, an open book or a lit electronic display screen (e.g., a smartphone, TV, or tablet screen) tend to be objects that a wearer focuses on. Objects may be detected in an image and, in particular embodiments, if an object is detected that corresponds to a predetermined set of objects that tend to capture a person's focus, then the region of the image that corresponds to such an object may be determined to correspond to the gaze of the wearer. In particular embodiments, object detection and the relative area of the region of the image that the object appears in may be used to estimate the gaze of the wearer. For example, an object that is in a central region of the image or even somewhat outside a central region may nevertheless be determined to be the object of focus, while such an object may not be estimated to correspond to the gaze of the wearer if the object is at the very periphery of the image. In addition or the alternative, particular embodiments may use the relative amount of the image occupied by an object to estimate the gaze of a wearer. For example, a TV that is very far away from the wearer may not be estimated to correspond to the gaze of the wearer, even if it is otherwise in the central vision.

In particular embodiments, estimating the gaze of the wearer of the device may include detecting the wearer's gaze. For example, an inward-facing camera or sensor (e.g., of a head-worn device) may track the wearer's gaze direction and/or focus, for example by tracking the wearer's pupils and/or irises, etc. The detected gaze may then be used to determine the area of the environment at which the wearer is looking, and the camera may then be focused to correspond to the wearer's detected gaze.

In particular embodiments, a wearer's estimated gaze may be represented as a gaze vector that identifies the direction and, in particular embodiments, the depth of the wearer's gaze.

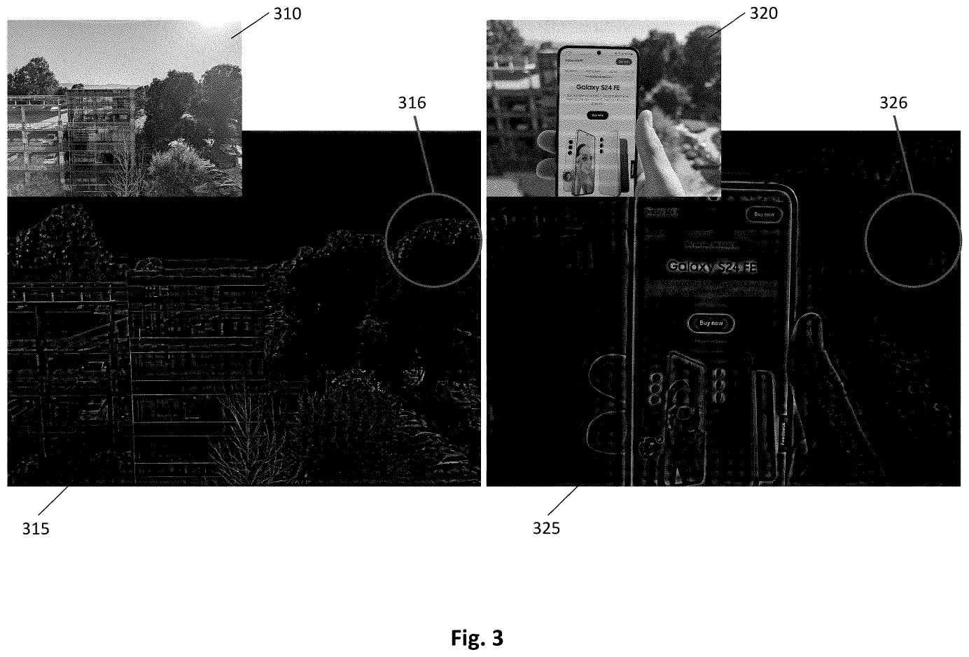

Step 120 of the example method of FIG. 1 includes focusing the camera based on the estimated gaze of the wearer. In other words, the user's estimated gaze (e.g., the portion of an initial image that is estimated to correspond to the wearer's gaze, or a detected gaze using eye tracking) is set as the focus of the camera at which subsequent images will be captured, at least until step 110 is repeated. For example, if the wearer's gaze is estimated to be a building in the example of FIG. 3, then the camera will focus on that region; on the other hand, if the wearer's gaze is estimated to be a mobile device in the example of FIG. 3, then the camera will focus on that (much closer) region. Any suitable autofocus techniques may be used to focus the camera on the region estimated to correspond to the gaze of the wearer.

By focusing the camera based on the estimated wearer's gaze, the focused image provides a good representation of the image at the wearer's retina. The representation can be particularly accurate in instances where the numerical aperture of the camera is similar to that of the eye. Thus, the brightness, contrast, spatial frequency, and other eye-health-related metrics extracted from the image provide a good representation of how those metrics are present on portions of the retina.

Step 130 of the example method of FIG. 1 includes capturing, by the focused camera, one or more focused images of the environment of the wearer. Images may be captured at a predetermined interval (e.g., every 1 minute, 5 minutes, 10 minutes, etc.) or may be captured relatively continuously (e.g., up to the refresh rate of the camera, although that may result in relatively higher power demands). Images may also or alternatively be captured when the wearer's gaze changes, e.g., based on detected movement of the device or of the wearer (e.g., as determined by a GPS sensor or other location-detection techniques) or based on visual attributes such as changes in brightness, etc. that indicate a change in scene.

Step 140 of the example method of FIG. 1 includes determining, from at least one of the one or more focused images of the environment of the wearer, one or more visual features of the environment of the wearer related to the eye health of the wearer. As explained below, this step can include heuristic approaches that identify specific predetermined visual features (e.g., content having a particular range of spatial frequencies) and approaches that use one or more trained AI models to predict visual features related to a wearer's eye health.

In particular embodiments, visual features related to a wearer's eye health can include time spent at near focus. This is determined as, for example, the time during which the camera's focus (which corresponds to the wearer's estimated gaze) is relatively in the foreground. In particular embodiments, visual features related to a wearer's eye health can include light intensity. For example, relatively higher light intensity is correlated with reduced myopia progression, and therefore the intensity of a captured image (e.g., as determined by the intensity of pixels in the image or in portions of the image) can be a visual feature that corresponds to the wearer's eye health.

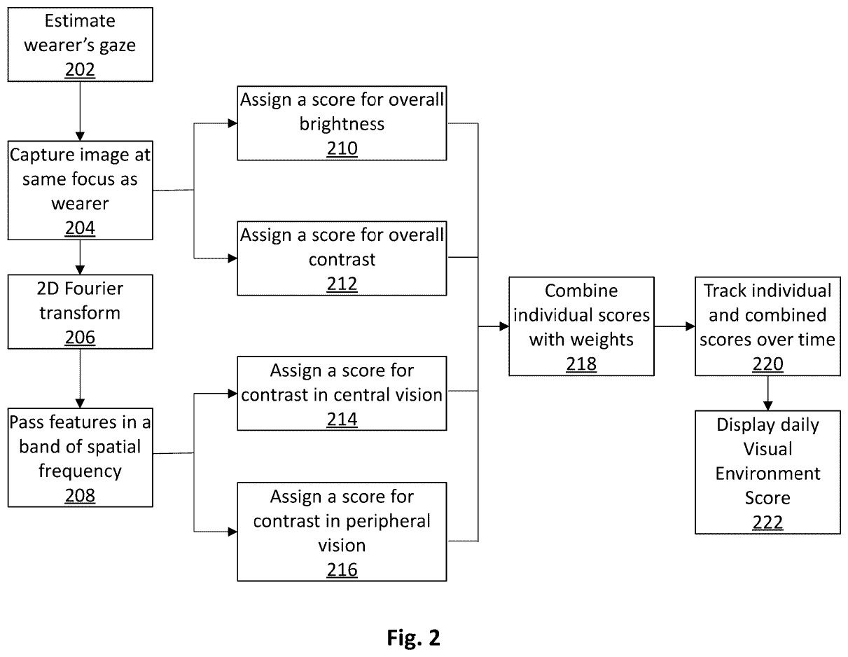

In particular embodiments, visual features related to a wearer's eye health can include the spatial frequency of content in environment. For instance, a lack of relatively high-frequency spatial content, which is due to the absence of relatively small features in a scene, is correlated with increased myopia progression. In other words, scenes that have relatively higher spatial-frequency content (such as those typically provided by outdoor, natural environments) within a particular range are correlated with reduced myopia progression. Particular embodiments, such as the example discussed in connection with FIG. 2, below, independently treat the spatial frequencies in the wearer's central vision compared to such features in the peripheral vision. More generally, particular embodiments may consider visual features in the central vision and in the peripheral vision separately.

FIG. 2 illustrates an example technique that implements the example method of FIG. 1, among other things, and illustrates an example of myopia-related visual features that may be determined in a heuristic-type approach. The example technique of FIG. 2 starts with estimating the wearer's gaze in step 202, for instance by using any of the techniques described above. The camera is focused based on the wearer's gaze in step 204. In the example technique of FIG. 2, step 206 includes performing a two-dimensional Fourier transform of the image content. As explained below, this transform is used to identify whether sufficiently high-frequency (non-uniform) spatial features are present at the wearer's focal distance, as viewing a scene that does not include such features is associated with increased risk of myopia.

Step 208 of the example of FIG. 2 includes passing the transformed image of step 206 through a band of spatial frequencies. In particular embodiments, the spatial frequency band is tuned to a particular eye-health diagnostic. For instance, for myopia a band with 0.5 to 5 cycles per degree may be used, as this band is associated with reduced myopia progression. Other bands may be used, in addition or in the alternative.

FIG. 3 illustrates an example of a Fourier transform of a particular image and filtering based on a band of spatial frequencies. Image 310 in FIG. 3 includes a building and other outdoor features (e.g., trees) that are in focus and correspond to the region that is estimated to correspond to a wearer's gaze. In contrast, image 320 includes the same objects but also includes a mobile device in the foreground. Because a wearer is estimated as gazing at the mobile device, the outdoor features are out of focus, while the mobile device is in focus.

Filtered image 315 results from passing image 310 through a Fourier transform, filtering the result using a band of spatial frequencies (e.g., 0.5 to 5 cycles per degree), and then transforming the filtered result back into the spatial domain for the purposes of illustration. In image 315, the peripheral vision (e.g., region 316) includes several high-frequency features. In contrast, filtered image 325, which results from filtering image 320 as describe above, includes little to no high-frequency content in the peripheral regions (i.e., outside of the central region that corresponds to the wearer's estimated gaze). For example, region 326 corresponds to region 316 in filtered image 315, yet region 326 contains almost no high-frequency content due to the fact that in image 320, the region corresponding to region 316 is out of focus.

The example above and in FIG. 3 uses a 2D Fourier transform to extract frequency information for spatial features in a band that corresponds to reducing myopia progression. An environment that lacks content in these spatial frequencies can therefore be classified as relatively myopia inducing. Other techniques for extracting and filtering the spatial frequency of content in an image, both in the central vision and in the peripheral vision, may be used along with or as an alternative to the Fourier transform.

In particular embodiments, a filtered image such as images 315 or 325 may be displayed to a wearer, for instance so that the wearer can understand which portions of their environment have spatial frequency characteristics that help to impede myopia progression.

The example of FIG. 2 assigns a score to each visual feature identified in that example. The scores can be qualitative (e.g., high, medium, low) or quantitative (e.g., a binary score, or any real-valued score (e.g., normalized between 0 and 1), etc.). For instance, in the example of FIG. 2 a score is assigned to the overall brightness of a focused image in step 210. Here, the brightness may be determined from pixels in the entire image, or from pixels in one or more regions of the image (e.g., in a region corresponding to the central vision, to the periphery, etc.). The score may be binary based on a brightness threshold, e.g., such that brightness values below the brightness threshold receive a score of “0” while brightness values above the brightness threshold receive a score of “1.” Other embodiments may convert the brightness of the image to one of a range of continuous score values. For instance, perceived brightness is often roughly proportional to the square root of true radiant flux, and this relationship may be modified by biological factors such as pupil size, processing in the brain, etc. Thus, the relationship between derived brightness values (e.g., radiant flux) and perceived brightness may be determined and used to generate a brightness score, in particular embodiments.

The example of FIG. 2 assigns a score to the overall contrast of a focused image in step 212. In this example, the contrast is of the image as a whole. Step 214 assigns a score to the contrast in the region of the image that corresponds to the wearer's central vision, while step 216 assigns a score to the contrast in the region of the image corresponding to the wearer's peripheral vision. In the example of FIG. 2, steps 214 and 216 are derived from the pass-band spatial frequencies in the transformed image of step 208, as described above. As described above with respect to step 210, the scores assigned in steps 212, 214, and 216 may be qualitative or quantitative, and may be binary or from a set of continuous real values.

In particular embodiments, scores assigned to various visual features may be weighted in order to arrive at an overall score that characterizes the impact of a visual environment on the wearer's eye health. For instance, in the example of FIG. 2, step 218 includes combining the individual scores in steps 210, 212, 214, and 216 using a set of weights for each score. As one example, the score 216 corresponding to the contrast in the peripheral vision may be weighted more highly than score 214 corresponding to contrast in the central vision, because a human eye has often has a much higher field of view than does a camera, and therefore the image content may omit content that is present on the retina. For example, if the camera has a field of view that is smaller than that of the eye, the image from the camera will overrepresent the content in the eye's central vision and underrepresent the content in the eye's peripheral vision. For instance, the area of the retina with a certain stimulus (proportional to myopia progression) tends to increase with the square of the angular field of view In this example, the peripheral content captured by the camera may be weighted more heavily than the central content captured by the camera in order to correct the misrepresentation. Particular embodiments may likewise weight the relative impact of, e.g., a brightness-based score with the impact of high-frequency spatial content in the image.

In particular embodiments, determining the central vision of a wearer vs. the peripheral vision may be based on the region of the image that corresponds to the estimated gaze of the wearer, e.g., using a gaze vector for the wearer. For instance, particular embodiments may define the central vision to be centered on the gaze vector plus an additional 2 degrees (roughly corresponding to the foveal vision) from that vector. Other embodiments may define the central vision to be centered on the gaze vector plus an additional 18 degrees (roughly corresponding to the macular central vision) from that vector. Particular embodiments may use any value between those measures to identify the region of the image that corresponds to the wearer's central vision, or may use other measures to define the central vision. Peripheral vision is the portion of the image that corresponds to content outside the central vision, up to a maximum that corresponds to the wearer's peripheral vision (e.g., up to about 180 degrees) in instances in which the camera has a field of view that exceeds that of the human retina.

While the example of FIG. 2 illustrates a particular example of a heuristic-based implementation of the method of FIG. 1, other approaches may use one or more trained AI models to determine eye-health related visual features. For example, a trained model such a neural network (e.g., a CNN) may receive as input a focused image (e.g., an image corresponding to step 204 of the example method of FIG. 2) and then predict eye-health-related visual features from that input. The predicted visual features may be a specific visual feature (e.g., a spatial frequency related to myopia) or may be an overall characterization (e.g., a score, classification, etc.) of one or more visual features related to eye health.

To train the AI model, training images of various visual scenes may be used along with ground-truth labels or scores for each of the images characterizing the effect of that image on eye health. For instance, training images may be displayed to a set of users, and the effect of each image on the thickness of the choroid in one or more of each user's eyes may then be measured, e.g., using optical coherence tomography (OCT), as a more practical heuristic for long-term intraocular length change. The ground-truth label or score may be the measured effect or may be a characterization of that effect, e.g., as determined by an expert. This training data may be provided to an AI model, e.g., a CNN, which after training can then estimate the corresponding effect of a visual image on a wearer's eye health during inference.

Once visual features related to the wearer's eye health are determined from one or more focused images, then those determinations can be used for a variety of downstream functionalities. For example, information about the wearer's current visual environment (e.g., as determined by the most recent image) may be provided to the wearer. As another example, information about the wearer's visual environment over time (e.g., over a time period such as an hour, a day, a week, etc.) may be provided to the wearer. As another example, suggestions or notifications regarding the wearer's eye health may be provided to the wearer, for instance to suggest that the wearer take a specific action to improve their visual environment. In particular embodiments, the determinations and any of the notifications described above may be provided to a health professional and/or recorded in the wearer's medical records, for instance so that the wearer's doctor can make informed diagnostic and treatment plans for the wearer's eye health over time.

The example of FIG. 2 illustrates an embodiment in which a wearer's visual-feature scores and combined scores are tracked over time in step 220. This example embodiment also provides a daily visual environment score for the wearer in step 222.

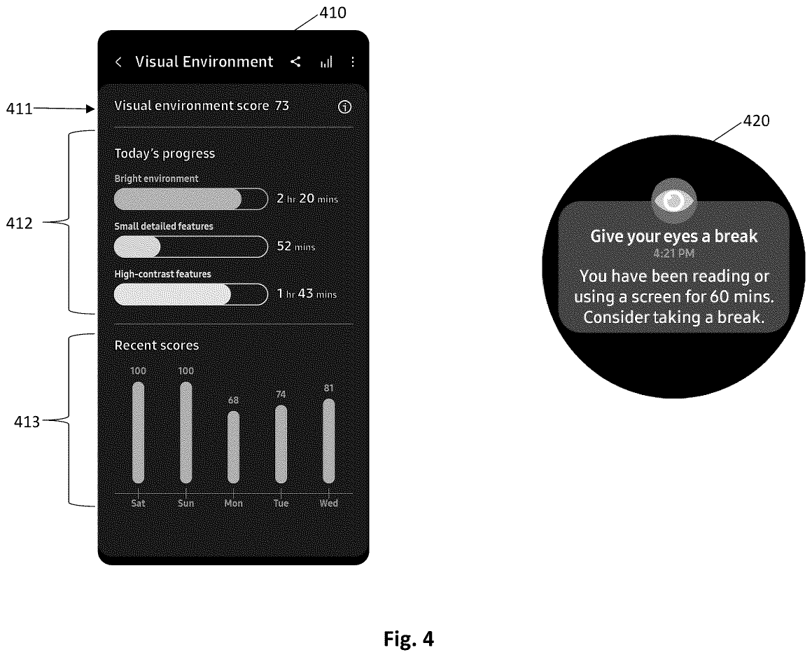

FIG. 4 illustrates example UIs that provides example eye-health related information determined from the wearer's visual environment. UI 410 may be displayed on, e.g., an application on a client device such as a smartphone, wearable device, personal computer, etc. In the example of FIG. 4, UI 410 provides a visual environment score 411 that scores the wearer's visual environment over a period of time, e.g., a day. For instance, this score may correspond to step 216 in the example of FIG. 2 or may be the output of a trained AI model. This score may reset each day and may range from 0 (poor visual environment) to 100 (ideal visual environment), in particular embodiments. Thus, the wearer may be able to quickly digest the eye-health-related impact of their determined visual environments they have experienced over a period of time.

In the example of FIG. 4, UI 412 includes an identification of particular visual features (e.g., brightness, high contrast, and feature size), as well as a characterization of each feature. The characterization may be over the same period of time (e.g., a day) as is visual environment score 411, or may be over a different period of time. In the example of FIG. 4, rather than providing a score for each individual features, UI 412 identifies the estimated amount of time that each feature is present in the wearer's visual environment. For instance, in the example of FIG. 4, the wearer has been estimated to be in a bright environment for 2 hours and 20 minutes, but is estimated to have been in an environment with small, detailed feature for only 52 minutes. As illustrated in FIG. 4, a UI such as UI 412 may include a progress bar that indicates a recommended or ideal amount of time for each visual feature, for example so that the wearer can try to view scenes that contain visual features that have been lacking in the wearer's environment.

UI 413 of FIG. 4 identifiers a wearer's visual environment score over a period of time; here, the score for each day over a 5-day period. Thus, the wearer can view their visual environment scores over time. In particular embodiments, a wearer may be able to navigate through historical visual environment scores and can adjust the scoring intervals and length of time over which to view those scores.

In particular embodiments, scores or other characteristics (e.g., the time scores of UI 412) may be weighted based on the quality of the visual feature. For example, spending 10 minutes in a medium-bright room may be counted as the equivalent of 7 minutes in a bright room, and the time credit (in the example of FIG. 4) may be updated accordingly.

FIG. 4 illustrates an example of a notification 420 that can be provided to a wearer based on the determined visual features that the wearer has viewed. Here, example notification 420 provides a suggestion (“give your eyes a break”) as well as some information about the wearer's visual environment that led to the suggestion (that the wearer has been reading or using a screen for an hour). A notification may be surfaced to a wearer on a client device, such as on a display of a head-worn device, a smartphone, a personal computer, etc. In particular embodiments, a notification may be triggered based on the wearer's current visual environment, based on the wearer's recent environment (e.g., in the past hour), based on the inferred activity of the wearer (e.g., that the wearer is viewing something in the foreground, such as a book or a screen), based on the wearer's more long-term history (e.g., that the wearer has had a week of low-quality visual environments), or any combination therefore.

In particular embodiments, information such as shown in UI 410 and/or notifications such as shown in notification 420 may be provided to a medical professional and/or placed in a wearer's medical records.

Particular embodiments may use sensors in addition to an outward-facing camera to capture images of the environment and, in particular embodiments, an inward-facing camera or sensor to determine a wearer's gaze. For example, in particular embodiments a head-worn device may include a GPS sensor, for example to estimate whether the wearer is inside or outside. As another example, particular embodiments may use one or more brightness sensors, such as one or more photodiodes, phototransistors, photoresistors, or other photon-sensitive sensors to detect the brightness of the visual environment, rather than (or in addition to) making that determination from an image captured by the camera. As another example, particular embodiments may use channel information from a color camera, e.g. using a Bayer filter, a sensor in tandem with a diffraction grating, or another optically dispersive element to sense the wavelength/spectrum of incident light. In particular embodiments, spatial frequency may be measured by sampling Fourier/K-space or by performing a calculation on a cartesian space image. Such sensors may be integrated with a device that contains the outward-facing camera (e.g., with a head-worn device) or may be deployed in one or more separate devices.

In particular embodiments, the method of FIG. 1 may be performed by the device worn by the wearer, e.g., by software running on a pair of glass or a head-mounted device. In other embodiments, some of the steps of the method of FIG. 1 may be offloaded to another computing device, such as a smartphone or personal computer of the wearer, or a server device. For example, a head-worn device may capture images, estimate a wearer's gaze, and determine visual features from the captured images. The head-worn device may also surface notifications and other myopia-related information to the wearer (as may other electronic devices, such as a smartphone, etc.). The head-worn device may communicate the visual features to another device, e.g., via a wireless connection, which may then score or evaluate the visual features and perform related functionality (e.g., prepare notifications for the wearer, update a medical professional or health system, etc.). The connected device can include a smartphone, a personal computer, a tablet, a server device, or a combination therefore.

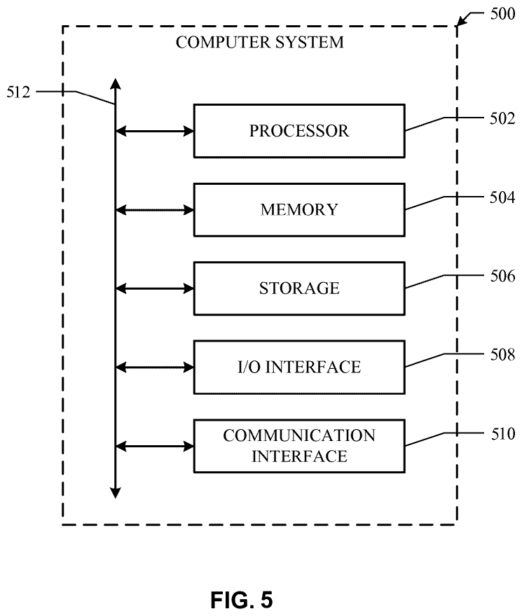

FIG. 5 illustrates an example computer system 500. In particular embodiments, one or more computer systems 500 perform one or more steps of one or more methods described or illustrated herein. In particular embodiments, one or more computer systems 500 provide functionality described or illustrated herein. In particular embodiments, software running on one or more computer systems 500 performs one or more steps of one or more methods described or illustrated herein or provides functionality described or illustrated herein. Particular embodiments include one or more portions of one or more computer systems 500. Herein, reference to a computer system may encompass a computing device, and vice versa, where appropriate. Moreover, reference to a computer system may encompass one or more computer systems, where appropriate.

This disclosure contemplates any suitable number of computer systems 500. This disclosure contemplates computer system 500 taking any suitable physical form. As example and not by way of limitation, computer system 500 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 500 may include one or more computer systems 500; be unitary or distributed; span multiple locations; span multiple machines; span multiple data centers; or reside in a cloud, which may include one or more cloud components in one or more networks. Where appropriate, one or more computer systems 500 may perform without substantial spatial or temporal limitation one or more steps of one or more methods described or illustrated herein. As an example and not by way of limitation, one or more computer systems 500 may perform in real time or in batch mode one or more steps of one or more methods described or illustrated herein. One or more computer systems 500 may perform at different times or at different locations one or more steps of one or more methods described or illustrated herein, where appropriate.

In particular embodiments, computer system 500 includes a processor 502, memory 504, storage 506, an input/output (I/O) interface 508, a communication interface 510, and a bus 512. Although this disclosure describes and illustrates a particular computer system having a particular number of particular components in a particular arrangement, this disclosure contemplates any suitable computer system having any suitable number of any suitable components in any suitable arrangement.

In particular embodiments, processor 502 includes hardware for executing instructions, such as those making up a computer program. As an example and not by way of limitation, to execute instructions, processor 502 may retrieve (or fetch) the instructions from an internal register, an internal cache, memory 504, or storage 506; decode and execute them; and then write one or more results to an internal register, an internal cache, memory 504, or storage 506. In particular embodiments, processor 502 may include one or more internal caches for data, instructions, or addresses. This disclosure contemplates processor 502 including any suitable number of any suitable internal caches, where appropriate. As an example and not by way of limitation, processor 502 may include one or more instruction caches, one or more data caches, and one or more translation lookaside buffers (TLBs). Instructions in the instruction caches may be copies of instructions in memory 504 or storage 506, and the instruction caches may speed up retrieval of those instructions by processor 502. Data in the data caches may be copies of data in memory 504 or storage 506 for instructions executing at processor 502 to operate on; the results of previous instructions executed at processor 502 for access by subsequent instructions executing at processor 502 or for writing to memory 504 or storage 506; or other suitable data. The data caches may speed up read or write operations by processor 502. The TLBs may speed up virtual-address translation for processor 502. In particular embodiments, processor 502 may include one or more internal registers for data, instructions, or addresses. This disclosure contemplates processor 502 including any suitable number of any suitable internal registers, where appropriate. Where appropriate, processor 502 may include one or more arithmetic logic units (ALUs); be a multi-core processor; or include one or more processors 502. Although this disclosure describes and illustrates a particular processor, this disclosure contemplates any suitable processor.

In particular embodiments, memory 504 includes main memory for storing instructions for processor 502 to execute or data for processor 502 to operate on. As an example and not by way of limitation, computer system 500 may load instructions from storage 506 or another source (such as, for example, another computer system 500) to memory 504. Processor 502 may then load the instructions from memory 504 to an internal register or internal cache. To execute the instructions, processor 502 may retrieve the instructions from the internal register or internal cache and decode them. During or after execution of the instructions, processor 502 may write one or more results (which may be intermediate or final results) to the internal register or internal cache. Processor 502 may then write one or more of those results to memory 504. In particular embodiments, processor 502 executes only instructions in one or more internal registers or internal caches or in memory 504 (as opposed to storage 506 or elsewhere) and operates only on data in one or more internal registers or internal caches or in memory 504 (as opposed to storage 506 or elsewhere). One or more memory buses (which may each include an address bus and a data bus) may couple processor 502 to memory 504. Bus 512 may include one or more memory buses, as described below. In particular embodiments, one or more memory management units (MMUs) reside between processor 502 and memory 504 and facilitate accesses to memory 504 requested by processor 502. In particular embodiments, memory 504 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 504 may include one or more memories 504, where appropriate. Although this disclosure describes and illustrates particular memory, this disclosure contemplates any suitable memory.

In particular embodiments, storage 506 includes mass storage for data or instructions. As an example and not by way of limitation, storage 506 may include a hard disk drive (HDD), a floppy disk drive, flash memory, an optical disc, a magneto-optical disc, magnetic tape, or a Universal Serial Bus (USB) drive or a combination of two or more of these. Storage 506 may include removable or non-removable (or fixed) media, where appropriate. Storage 506 may be internal or external to computer system 500, where appropriate. In particular embodiments, storage 506 is non-volatile, solid-state memory. In particular embodiments, storage 506 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 506 taking any suitable physical form. Storage 506 may include one or more storage control units facilitating communication between processor 502 and storage 506, where appropriate. Where appropriate, storage 506 may include one or more storages 506. Although this disclosure describes and illustrates particular storage, this disclosure contemplates any suitable storage.

In particular embodiments, I/O interface 508 includes hardware, software, or both, providing one or more interfaces for communication between computer system 500 and one or more I/O devices. Computer system 500 may include one or more of these I/O devices, where appropriate. One or more of these I/O devices may enable communication between a person and computer system 500. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 508 for them. Where appropriate, I/O interface 508 may include one or more device or software drivers enabling processor 502 to drive one or more of these I/O devices. I/O interface 508 may include one or more I/O interfaces 508, where appropriate. Although this disclosure describes and illustrates a particular I/O interface, this disclosure contemplates any suitable I/O interface.

In particular embodiments, communication interface 510 includes hardware, software, or both providing one or more interfaces for communication (such as, for example, packet-based communication) between computer system 500 and one or more other computer systems 500 or one or more networks. As an example and not by way of limitation, communication interface 510 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 510 for it. As an example and not by way of limitation, computer system 500 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 500 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 500 may include any suitable communication interface 510 for any of these networks, where appropriate. Communication interface 510 may include one or more communication interfaces 510, where appropriate. Although this disclosure describes and illustrates a particular communication interface, this disclosure contemplates any suitable communication interface.

In particular embodiments, bus 512 includes hardware, software, or both coupling components of computer system 500 to each other. As an example and not by way of limitation, bus 512 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 512 may include one or more buses 512, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

This disclosure contemplates a system that includes one or more non-transitory computer readable storage media storing instructions; and one or more processors coupled to the one or more non-transitory computer readable storage media and operable to execute the instructions to perform certain functions includes embodiments in which those functions are performed by a single processor, embodiments in which those functions are performed by multiple processors that each perform all the functions, and embodiments in which those functions are performed by multiple processors (e.g., in separate computing devices) where each processor performs at least one function but less than all recited functions.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
estimating a gaze of a wearer of a device comprising an outward facing camera;
focusing the camera based on the estimated gaze of the wearer;
capturing, by the focused camera, one or more focused images of an environment of the wearer; and
determining, from at least one of the one or more focused images of the environment of the wearer, one or more visual features of the environment of the wearer related to the eye health of the wearer.

2. The method of claim 1, wherein the one or more visual features of the environment of the wearer related to the eye health of the wearer comprises one or more myopia-inducing visual features.

3. The method of claim 2, wherein the device comprises a head-worn device.

4. The method of claim 3, wherein estimating the gaze of the wearer of the device comprises:
capturing, by the outward facing camera of the device, an initial image of the environment of the wearer;
estimating a portion of the initial image that corresponds to the gaze of the wearer.

5. The method of claim 4, wherein estimating a portion of the initial image that corresponds to a gaze of the wearer comprises estimating a central region of the initial image as corresponding to the gaze of the wearer.

6. The method of claim 4, wherein estimating a portion of the initial image that corresponds to a gaze of the wearer comprises detecting one or more objects in the initial image.

7. The method of claim 3, wherein estimating the gaze of the wearer comprises:
tracking, by an inward facing camera, one or more eyes of the wearer; and
identifying at least a direction of the wearer's gaze based on the tracked one or more eyes of the wearer.

8. The method of claim 3, wherein the myopia-inducing visual features comprise one or more of (1) a brightness of a region of the wearer's central vision in the one or more focused images (2) a brightness of a region outside of the wearer's central vision in the one or more focused images (3) a contrast of the one or more focused images (4) a contrast of a region of the wearer's central vision in the one or more focused images or (5) a contrast of a region outside of the wearer's central vision in the one or more focused images.

9. The method of claim 8, further comprising:
for each focused image, filtering features in the focused image in a predetermined band of spatial frequencies; and
determining, from the filtered features of each focused image, one or more of (1) the contrast of the region of the wearer's central vision in that focused image or (2) the contrast of the region outside of the wearer's central vision in that focused image.

10. The method of claim 8, further comprising assigning a score to each of the determined myopia-inducing visual features.

11. The method of claim 10, further comprising determining an overall myopia-inducing score by combining a weighted score of each of a plurality of myopia-inducing visual features.

12. The method of claim 3, further comprising providing, for presentation on a display of an electronic device, a characterization of the myopia-inducing visual features of the environment of the wearer.

13. The method of claim 12, wherein the characterization comprises an amount of time spent by the wearer in an environment that reduces myopia incidence.

14. The method of claim 3, further comprising tracking the myopia-inducing visual features in the environment of the wearer over time.

15. The method of claim 3, further comprising providing, for the wearer, a notification comprising an identification one or more actions for the wearer to take based on the myopia-inducing visual features of the environment.

16. A system comprising:
one or more non-transitory computer readable storage media storing instructions; and one or more processors coupled to the one or more non-transitory computer readable storage media and operable to execute the instructions to:
estimate a gaze of a wearer of a device comprising an outward facing camera;
focus the camera based on the estimated gaze of the wearer;
capture, by the focused camera, one or more focused images of an environment of the wearer; and
determine, from at least one of the one or more focused images of the environment of the wearer, one or more visual features of the environment of the wearer related to the eye health of the wearer.

17. The system of claim 16, wherein the one or more visual features of the environment of the wearer related to the eye health of the wearer comprises one or more myopia-inducing visual features.

18. The system of claim 17, wherein the device comprises a head-worn device.

19. One or more non-transitory computer readable storage media storing instructions that are operable when executed by one or more processors to:
estimate a gaze of a wearer of a device comprising an outward facing camera;
focus the camera based on the estimated gaze of the wearer;
capture, by the focused camera, one or more focused images of an environment of the wearer; and
determine, from at least one of the one or more focused images of the environment of the wearer, one or more visual features of the environment of the wearer related to the eye health of the wearer.

20. The media of claim 19, wherein:
the one or more visual features of the environment of the wearer related to the eye health of the wearer comprises one or more myopia-inducing visual features; and
the device comprises a head-worn device.
